(19) United States
(12) Patent Application Publication
(10) Pub. No.: US 2019/0091380 A1
(43) Pub. Date: Mar. 28, 2019

(54) COPOLYMER, ANTITHROMBOTIC COATING AGENT USING SAME AND MEDICAL DEVICE

(71) Applicants: Maruzen Petrochemical Co., Ltd., Chuo-ku (JP); National University Corporation Yamagata University, Yamagata-shi (JP)

(72) Inventors: Masaru TANAKA, Fukuoka (JP); Norihiro YOSHIDA, Chiba (JP)

(73) Assignees: Maruzen Petrochemical Co., Ltd., Chuo-ku (JP); National University Corporation Yamagata University, Yamagata-shi (JP)

(21) Appl. No.: 16/080,563

(22) PCT Filed: Jan. 24, 2017
(86) PCT No.: PCT/JP2017/002199

§ 371 Date: Aug. 28, 2018

(30) Foreign Application Priority Data
Feb. 29, 2016 (JP) 2016-037277

(51) Int. Cl. A61L 33/06; C08F 216/18

(52) U.S. Cl. CPC A61L 33/064; C08F 216/18; A61L 2300/42; A61L 2300/606; A61L 2420/00

(57) ABSTRACT

A biocompatible material, which has excellent film formation properties and resistance to water dissolution, and is easily applied on various bases as a coating, while having excellent antithrombotic properties, and to provide an antithrombotic coating agent and an antithrombotic coating film produced by using the biocompatible material, and a medical device provided with the antithrombotic coating film. A copolymer, containing at least one repeating unit (A) represented by formula (1) (wherein, n represents an integer of 2 to 10 and R¹ represents a methyl group or an ethyl group), and at least one repeating unit (B) represented by formula (2) (wherein R² represents an aliphatic hydrocarbon group); an antithrombotic coating agent containing the copolymer and an organic solvent; an antithrombotic coating film formed of the copolymer; and a medical device provided with the coating film.

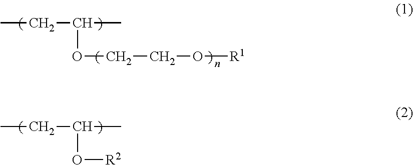

### TECHNICAL FIELD

The present invention relates to a copolymer and an antithrombotic coating agent produced by using the copolymer, and particularly to a copolymer that can provide excellent antithrombotic properties, an antithrombotic coating agent containing the copolymer, an antithrombotic coating film formed of the copolymer, and a medical device provided with the coating film.

### BACKGROUND ART

It is essential for medical devices used in contact with blood, such as catheters, guide wires, stents, blood vessel prostheses, vascular bypass tubes, prosthetic valves, blood filters, plasmapheresis devices, artificial organs, oxygenator devices, dialyzers, blood transfusion apparatuses, blood circuits, and blood bags, to have antithrombotic properties to prevent coagulation of blood. Thus, it is desirable to develop an antithrombotic coating agent that provides excellent antithrombotic properties to surfaces of bases used in the medical devices.

An example of known biocompatible materials having antithrombotic properties is poly(2-methoxyethyl acrylate) (PMEA) (see PTL 1). PMEA is known to have biocompatibility, such as antithrombotic properties, due to its so-called intermediate water (water in the state where an exothermic peak due to low temperature crystal formation of water is stably observed around −40° C. in the course of heating from −100° C.) which is considered as water that is weakly bound to a polymer chain by an interaction with the polymer chain.

However, since PMEA itself is a hydrophilic polymer, there are concerns about peeling or dissolution in use. Thus, in PTL 1, alkyl (meth)acrylate which is more hydrophobic than 2-methoxyethyl acrylate is copolymerized to the extent that the intermediate water is sufficiently present (9 to 17% by mole in Examples).

As a technique for improving the film formation properties of PMEA, a thin film of a polymer blend in which poly(methyl methacrylate) (PMMA) is mixed with PMEA is reported (see PTL 2). According to PTL 2, the polymer blend thin film that has a prescribed proportion of PMEA shows superior antithrombotic properties to pure PMEA.

However, in such methods of improving the hydrophilicity and film formation properties by introducing PMMA or other hydrophobic components through copolymerization, polymer blending, or other conventional techniques, the amount of the hydrophobic component cannot be increased since biocompatibility is inhibited as the amount increases, which has limited the effect of improvement.

In addition, adhesion of proteins, cells, or other biocomponents is liable to occur when a hydrophobic component is introduced, and therefore such a hydrophobic component has not been able to be used in applications where anti-fouling properties (suppression of protein adsorption and suppression of cell adhesion) are required.

On the other hand, poly(2-methoxyethyl vinyl ether) (PMOVE) is known as a biocompatible material which has an oxyethylene chain structure in a side chain and has intermediate water like PMEA, and is reported to have superior antithrombotic properties to PMEA (see PTL 3).

However, PTL 3 discloses only PMOVE as a specific example with antithrombotic properties, and does not disclose antithrombotic properties of any polymer obtained by extending the oxyethylene chain which constitutes a side chain of the vinyl ether polymer or a copolymer with a hydrophobic comonomer. Moreover, anti-fouling properties in the vinyl ether polymers were never studied.

PMOVE, which is a highly viscous oil substance and is also soluble in water at living body temperature, is difficult to be used as an antithrombotic coating agent as it is. For this reason, in PTL 3, PMOVE applied on a base is irradiated with a gamma ray to crosslink and insolubilize the PMOVE. However, such a method for forming a film is not a common method and has a problem in that the material, shape, and form of the base to be coated are limited.

As another method for forming a thin film containing PMOVE, a method in which 2-methoxyethyl vinyl ether (MOVE) is copolymerized with a vinyl ether having an alicyclic backbone such as tricyclodecane vinyl ether (TCDVE) is proposed (see PTL 4). However, in Examples of PTL 4, examples of formation of a thin film are illustrated only for a polymer having a short oxyethylene chain as a side chain, such as PMOVE and poly(2-ethoxyethyl vinyl ether) (PEOVE), and furthermore, the biocompatibility, antithrombotic properties, and anti-fouling properties of the copolymer were never evaluated.

### CITATION LIST

### Patent Literature

PTL 1: JP-A-2004-161954

PTL 2: JP-A-2013-121430

PTL 3: JP-A-2014-47347

PTL 4: Japanese Patent No. 4528601

### SUMMARY OF INVENTION

### Technical Problem

The present invention is made in view of the above situation, and has an object to find a biocompatible material that has excellent film formation properties and resistance to water dissolution and can be easily applied on various bases as a coating, while having excellent antithrombotic properties, and to provide an antithrombotic coating agent and an antithrombotic coating film produced by using the same, and a medical device provided with the antithrombotic coating film. The present invention also has an object to provide a biocompatible material which is also excellent in anti-fouling properties and a medical device produced by using the same.

### Solution to Problem

In general, copolymerization of a hydrophobic comonomer having a hydrophobic group leads to increase of hydrophobic interactions with biocomponents to cause adhesion of platelets and adsorption modification and activation of proteins in conditions of contact with proteins in biotissues and blood. For this reason, there has not been any example in which a polymer containing a hydrophobic vinyl ether is studied as a biocompatible material, and any copolymer with a vinyl ether having an oxyethylene chain structure, such as MOVE, has also not been studied as a biocompatible material.

However, as a result of intensive studies of copolymers of a vinyl ether containing an oxyethylene chain structure with a hydrophobic vinyl ether, the present inventors have found that a copolymer of a vinyl ether having a repetition number of oxyethylene chains of two or more with a aliphatic vinyl ether has excellent film formation properties and resistance to water dissolution, and surprisingly, also has excellent antithrombotic properties and anti-fouling properties, completing the present invention.

Specifically, the present invention is as follows.

(1) A copolymer containing at least one repeating unit (A) represented by the following formula (1):

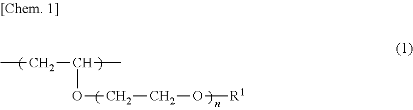

(wherein, R¹ represents a methyl group or an ethyl group, and n represents an integer of 2 to 10), and at least one repeating unit (B) represented by the following formula (2):

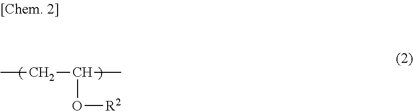

(wherein, R² represents an aliphatic hydrocarbon group).

(2) The copolymer according to the above (1), wherein the composition ratio (molar ratio) of the repeating unit (A) to the repeating unit (B) is 90/10 to 1/99.

(3) The copolymer according to the above (1) or (2), wherein the group R² in the repeating unit (B) is a linear or branched alkyl group or alkenyl group having 2 to 10 carbon atoms, or a monocyclic or polycyclic alkyl group or alkenyl group having 5 to 15 carbon atoms.

(4) The copolymer according to the above (1) or (2), wherein the group R² in the repeating unit (B) is a linear or branched alkyl group or alkenyl group having 2 to 10 carbon atoms.

(5) A biocompatible material containing the copolymer according to the above (1) or (2).

(6) An antithrombotic coating agent containing the copolymer according to any one of the above (1) to (4) and an organic solvent.

(7) The antithrombotic coating agent according to the above (6), wherein the organic solvent is one or two or more selected from the group consisting of an aromatic hydrocarbon, an aliphatic hydrocarbon, a halogenated hydrocarbon, an ether, an alcohol, a ketone, and an ester.

(8) The antithrombotic coating agent according to the above (6) or (7), wherein the amount of the copolymer is 0.05 to 10 parts by mass and the amount of the organic solvent is 99.95 to 90 parts by mass.

(9) An antithrombotic coating film formed of the copolymer according to any one of the above (1) to (4).

(10) A medical device provided with an antithrombotic coating film formed of the copolymer according to any one of the above (1) to (4).

(11) The medical device according to the above (10), wherein a part or the whole of a section to be in contact with blood is coated with the antithrombotic coating film.

(12) The medical device according to the above (10) or (11), which is any one of an intracorporeal implant type artificial organum or therapeutic implement, an extracorporeal circulation type artificial organ, catheter, blood vessel prosthesis, vascular bypass tube, prosthetic valve, blood filter, plasmapheresis device, or blood transfusion apparatus, or an extracorporeal circuit for blood.

### Advantageous Effects of Invention

The copolymer of the present invention can be easily applied on various bases as a coating, has excellent film formation properties and resistance to water dissolution, and can form an antithrombotic coating film. The copolymer can be suitably used as a biocompatible material.

Accordingly, the copolymer is useful as a component of an antithrombotic coating agent, and an antithrombotic coating film formed of the copolymer has excellent antithrombotic properties and can significantly suppress coagulation of blood even when it is in contact with blood over a long period of time. Thus, the copolymer is highly useful for a coating film of a medical device.

A coating film formed of the copolymer of the present invention can prevent adhesion of biocomponents, such as cells, and can be suitably used in various medical devices as a material having anti-fouling properties.

### DESCRIPTION OF EMBODIMENTS

### <Copolymer>

The copolymer of the present invention contains at least one repeating unit (A) represented by the formula (1) and at least one repeating unit (B) represented by the formula (2) and thereby exhibits excellent biocompatibility, such as antithrombotic properties and anti-fouling properties.

Examples of monomers providing the repeating unit (A) of the formula (1) include hydrophilic vinyl ethers represented by the following formula (3):

[Chem. 3]

CH₂═CH—OCH₂—CH₂—OₙR¹ (3)

wherein R¹ is a methyl group or ethyl group, the repetition number n of the oxyethylene chains is an integer of 2 to 10, preferably an integer of 2 to 6, more preferably an integer of 2 to 4, and particularly preferably 2 or 3.

Specific examples of hydrophilic vinyl ethers represented by the formula (3) include 2-(2-methoxyethoxy)ethyl vinyl ether (synonym: diethylene glycol monomethyl monovinyl ether), 2-(2-ethoxyethoxy)ethyl vinyl ether (synonym: diethylene glycol monoethyl monovinyl ether; hereinafter referred to as “EOEOVE”), 2-[2-(2-methoxyethoxy)ethoxy]ethyl vinyl ether (synonym: triethylene glycol monomethyl monovinyl ether; hereinunder referred to as “TEGVE”), and 2-[2-(2-ethoxyethoxy) ethoxy]ethyl vinyl ether (synonym: triethylene glycol monoethyl monovinyl ether). Among them, EOEOVE and TEGVE are preferred in terms of film formation properties and resistance to water dissolution, and EOEOVE is particularly preferred due to its superior antithrombotic properties.

Examples of monomers providing the repeating unit (B) include hydrophobic vinyl ethers represented by the following formula (4)

[Chem. 4]

CH₂—CH—OR² (4)

wherein R² is an aliphatic hydrocarbon group, and specifically a linear or branched alkyl group or alkenyl group or a monocyclic or polycyclic alkyl group or alkenyl group.

The linear or branched alkyl group or alkenyl group preferably has 2 to 10 carbon atoms, more preferably has 2 to 8 carbon atoms, and further preferably has 2 to 6 carbon atoms.

Specific examples of linear or branched alkyl groups or alkenyl groups include linear or branched alkyl groups, such as an ethyl group, an n-propyl group, an isopropyl group, an n-butyl group, an isobutyl group, a tert-butyl group, a 1-pentyl group, a 2-pentyl group, a 3-pentyl group, a 1-(2-methyl)-butyl group, a 2-(2-methyl)-butyl group, a 1-(3-methyl)-butyl group, a 2-(3-methyl)-butyl group, a (2,2-dimethyl)-propyl group, a 1-hexyl group, a 2-hexyl group, a 3-hexyl group, a 1-heptyl group, a 2-heptyl group, a 3-heptyl group, a 4-heptyl group, a 1-octyl group, and a 1-(2-ethyl)-hexyl group; and linear or branched alkenyl groups, such as a vinyl group, a 1-propenyl group, an allyl group, a 2-butenyl group, a 3-butenyl group, an isopropenyl group, an isobutenyl group, a 1-pentenyl group, a 2-pentenyl group, a 3-pentenyl group, a 4-pentenyl group, a 1-hexenyl group, a 2-hexenyl group, a 3-hexenyl group, a 4-hexenyl group, and a 5-hexenyl group.

The monocyclic or polycyclic alkyl group or alkenyl group preferably has 3 to 25 carbon atoms, more preferably has 4 to 20 carbon atoms, and further preferably 5 to 15 carbon atoms.

Specific examples of monocyclic or polycyclic alkyl groups or alkenyl groups include monocyclic alkyl groups or alkenyl groups, such as a cyclopentyl group, a cyclopentylmethyl group, a methylcyclopentyl group, a dimethylcyclopentyl group, a cyclohexyl group, a cyclohexylmethyl group, a methylcyclohexyl group, a dimethylcyclohexyl group, a cyclohexenyl group, a cycloheptyl group, a cyclooctyl group, a cyclononyl group, a cyclodecyl group, a cycloundecyl group, a cyclododecyl group, a cyclotridecyl group, a cyclotetradecyl group, a cyclopentadecyl group, a cyclooctadecyl group, and a cycloicosyl group; and polycyclic alkyl groups or alkenyl, such as a bicyclohexyl group, a decahydronaphthyl group, a norbornyl group, a methylnorbornyl group, an isobornyl group, an adamantyl group, a tricyclodecanyl group, a tricyclodecenyl group, and a tetracyclododecyl group.

Among the aliphatic hydrocarbon groups, an n-butyl group and a tricyclodecanyl group are preferred in terms of film formation properties, resistance to water dissolution, and antithrombotic properties, and an n-butyl group is particularly preferred due to its superior antithrombotic properties.

The copolymer of the present invention can be prepared by polymerizing the hydrophilic vinyl ether (3) and the hydrophobic vinyl ether (4) according to an ordinary method. As a polymerization method, living cation polymerization is particularly preferred for obtaining a copolymer having a desired composition ratio and molecular weight in a highly reproducible manner. Since the molecular weight of a copolymer is determined substantially uniquely by the molar ratio of a monomer and a polymerization initiator in a living cation polymerization method, the molecular weight of the copolymer can be controlled arbitrarily over a wide range by varying the amount of monomers used and the amount of a polymerization initiator used.

Any polymerization initiator that can promote cation polymerization in a living manner can be used in living cation polymerization without any limitation. For example, HI/I₂-type initiators (for example, JP-A-60-228509), and polymerization initiators obtained by combining a Lewis acid catalyst (an organic aluminum compound or the like) and an additive, such as a base (ether, ester, or the like) (for example, Japanese Patent No. 3096494, JP-B-7-2805, JP-A-62-257910, JP-A-1-108202, and JP-A-1-108203) are suitably used as a living cation polymerization initiator for vinyl ethers.

The amount of a polymerization initiator used is preferably 0.001 to 20% by mole based on the total amount of the raw monomers, more preferably 0.01 to 10% by mole, and particularly preferably 1% by mole or less.

The living cation polymerization reaction is preferably performed in the presence of an appropriate organic solvent, but may be performed in the absence of an organic solvent. Examples of usable organic solvents include aromatic hydrocarbon solvents, such as benzene, toluene, and xylene; aliphatic hydrocarbon solvents, such as propane, n-butane, isobutane, n-pentane, isopentane, n-hexane, n-heptane, n-octane, isooctane, decane, hexadecane, and cyclohexane; halogenated hydrocarbon solvents, such as methylene chloride, ethylene chloride, and carbon tetrachloride; ether solvents, such as diethyl ether, dibutyl ether, tetrahydrofuran (THF), dioxane, and ethylene glycol diethyl ether. The organic solvents may be used alone or in combination of two or more thereof as needed. Among the organic solvents, hydrocarbon solvents, such as aromatic hydrocarbon solvents and aliphatic hydrocarbon solvents, are preferred, and toluene or cyclohexane is particularly preferred.

The polymerization temperature in the polymerization reaction varies depending on the types of the polymerization initiator, monomer, solvent used and the like, but is generally −80 to 150° C., preferably −50 to 100° C., and particularly preferably −20 to 80° C. The polymerization time varies depending on the polymerization initiator, monomer, solvent, reaction temperature used, but is generally approximately 10 minutes to 100 hours. The polymerization reaction can be suitably performed by any of a batch method and a continuous method. After the polymerization reaction, a purification treatment may be performed according to a known method to remove unreacted monomers as needed.

The composition ratio (molar ratio) of the repeating unit (A) to the repeating unit (B) in the copolymer of the present invention can be arbitrarily selected in the range where the film formation properties and antithrombotic properties are not impaired, but surprisingly in the copolymer of the present invention, a copolymer having a lower proportion of the repeating unit (A) which is a hydrophilic unit exhibits higher antithrombotic properties. Accordingly, the composition ratio (molar ratio) of the repeating unit (A) to the repeating unit (B) is preferably in the range of 90/10 to 1/99, more preferably in the range of 70/30 to 3/97, and particularly preferably in the range of 50/50 to 5/95.

The molecular weight of the copolymer in the present invention is approximately 1,000 to 1,000,000, preferably 2,000 to 500,000, and more preferably 3,000 to 300,000 as a weight average molecular weight (Mw) determined, for example, from a standard polystyrene calibration curve by gel permeation chromatography (GPC).

The ratio (Mw/Mn) of the weight average molecular weight (Mw) to the number average molecular weight (Mn) of the copolymer of the present invention is preferably 1.0 to 5.0, more preferably 1.0 to 3.0, and particularly preferably 1.0 to 1.5. Such Mw and Mw/Mn in the above ranges provide a copolymer having excellent coating performance and excellent antithrombotic properties.

The copolymer of the present invention is obtained by copolymerizing the hydrophilic vinyl ether and the hydrophobic vinyl ether as described above. There is no limitation in the sequencing manner of the monomer units, and the copolymer may be any of a random copolymer and a block copolymer. Examples of block copolymers include block polymers, such as a di-block type (A-B), a tri-block type (A-B-A or B-A-B), and a multi-branched star type ([B-A]ₙ, [A-B]ₙ, or AₙBₘ; wherein n and m are numbers of branches). From the viewpoint of uniformly dispersing the hydrophilic units, random copolymers are preferred.

### <Antithrombotic Coating Agent>

Since the copolymer of the present invention obtained as described above has excellent biocompatibility, such as antithrombotic properties and anti-fouling properties, the copolymer can be suitably used as a biocompatible material and, for example, can be found into an antithrombotic coating agent containing the copolymer as an active constituent. The antithrombotic coating agent can be prepared by blending an appropriate solvent with the copolymer.

The type and concentration of the solvent in the antithrombotic coating agent can be appropriately selected according to the composition and molecular weight of the copolymer and the type and surface properties of the base to be coated.

As a solvent in the antithrombotic coating agent, for example, organic solvents mentioned as a polymerization solvent in living cation polymerization can be used. In addition, alcohol solvents, such as methanol, ethanol, and isopropanol; ketone solvents, such as acetone, methyl ethyl ketone, and methyl amyl ketone; and ester solvents, such as methyl acetate, ethyl acetate, amyl acetate, and ethyl lactate can also be suitably used. Among the organic solvents, hydrocarbon solvents, such aromatic hydrocarbon solvents and aliphatic hydrocarbon solvents, ether solvents, and mixed solvents thereof are preferred, and toluene, cyclohexane, THF, and mixed solvents thereof are particularly preferred. The organic solvents may be used alone or in combination of two or more thereof as needed.

The concentration of the solvent in the antithrombotic coating agent is preferably 99.95 to 90 parts by mass of the solvent relative to 0.05 to 10 parts by mass of the copolymer.

The antithrombotic coating agent described above is used for forming an antithrombotic coating film containing the copolymer on various bases, in particular, on bases that may be brought into contact with blood.

The method for forming an antithrombotic coating film using the antithrombotic coating agent is appropriately selected from, but not limited to, known methods, such as application methods, spraying methods, dipping methods, spin coating methods according to the material, shape, and form of the base. The antithrombotic coating film can be formed by a simple operation in which, for example, a base is immersed in an antithrombotic coating agent that contains the copolymer and an organic solvent, and then the coating is dried by air or dried with heating.

Since the antithrombotic coating agent of the present invention has a simple configuration as described above, there is no limitation in the material, shape, and form of the base on which the antithrombotic coating film is formed, and the antithrombotic coating agent can be used on a base of any shape or form, such as a film, sheet, plate, fiber, nonwoven fabric, porous body, tube, hollow yarn or fiber, particle, and powder.

Examples of materials of the base include synthetic resins, such as polyolefin, for example, polypropylene and polyethylene, nylon, polyester, polyacrylonitrile, halogenated polyolefin, polystyrene, polycarbonate, polyvinyl chloride, polyurethane, polyamide, polysulfone, polyethersulfone, poly(meth)acrylate, an ethylene-vinyl alcohol copolymer, and a butadiene-acrylonitrile copolymer, and blend polymers thereof; natural polymers, such as cotton and hemp; inorganic materials, such as metals, ceramics, and glasses; and composite materials thereof, and these materials can be used.

Preferred examples of objects on which an antithrombotic coating film is formed from the antithrombotic coating agent of the present invention include medical devices. Many medical implements are brought into contact with blood, and in such a case, adhesion of platelets and coagulation of blood by aggregation have to be prevented, and therefore such medical implements are required to be provided with an antithrombotic coating film. Accordingly, in the medical devices, a part, and preferably, the whole of a section to be in contact with blood is preferably coated with the antithrombotic coating film.

The medical device of the present invention which is treated with an antithrombotic coating agent as described above is provided with the antithrombotic coating film, and thus can be suitably used particularly for applications in which a medical device is used in direct contact with blood. Specifically, the medical devices can be used for various applications, such as intracorporeal implant type artificial organa or therapeutic implements, extracorporeal circulation type artificial organs, catheters (for example, cardiovascular catheters, such as angiography catheters, guide wires, and PTCA catheters, digestive organ catheters and tubes, such as gastric tube catheters, gastrointestinal catheters, and esophageal tubes, and urological catheters, such as urethral catheters and ureteral catheter), blood vessel prostheses, vascular bypass tubes, prosthetic valves, blood filters, plasmapheresis devices, blood transfusion apparatuses, extracorporeal circuits for blood, blood bag, hemostatics, and biotissue adhesion materials. An antithrombotic coating film, which is formed on a part or the whole of a section of a medical device to be in contact with blood, can be used as a method for preventing formation of thrombi.

### EXAMPLES

The present invention will be described more specifically with reference to Examples and Synthetic Examples, but the present invention is never limited to the examples. In the examples, the composition ratios of copolymers were determined from ¹H-NMR analysis results, and the weight average molecular weights (Mw) and molecular weight distributions (Mw/Mn) thereof were determined from molecular weight analysis results (in terms of polystyrene) in GPC. The analysis apparatuses and measurement conditions are as follows.

### (NMR)

Apparatus: AVANCE400 manufactured by Burker
Solvent: deuterated acetone
Measurement temperature: 30° C.

### (GPC)

Apparatus: “HLC-8320GPC” manufactured by TOSOH Corporation
Detector: RI detector
Mobile phase: tetrahydrofuran
Flow rate: 1 mL/min
Column: 3× “Shodex LF-804” manufactured by SHOWA DENKO K.K.
Column temperature: 40° C.

### Synthetic Example 1

### Synthesis of N-Butyl Vinyl Ether/Diethylene Glycol Monoethyl Monovinyl Ether Random Copolymer (NBVE-Ran-EOEOVE):

Into a 300-mL three neck flask with a three-way stopcock previously subjected to dewatering with heat at 300° C. under dry nitrogen atmosphere for 10 minutes, 181 mL of toluene as a solvent, 76.4 mL of ethyl acetate as an added base, 4.0 mL of diethylene glycol monoethyl monovinyl ether (EOEOVE) as a hydrophilic vinyl ether, 28.2 mL of N-butyl vinyl ether (NBVE) as a hydrophobic vinyl ether, 4 mM (0.45 mL) of an acetic acid adduct of isobutyl vinyl ether as an initiator species were added, and the mixture was stirred well.

Next, the flask was kept at 0° C., and 8 mM (8.8 mL) of Et_{1.5}AlCl_{1.5} was added as a Lewis acid catalyst to start polymerization, and a reaction was carried out for 90 minutes.

The polymerization was stopped by methanol containing a small amount of sodium methoxide (1 M). To the solution in which the reaction stopped, 5% by mass of an ion exchange resin [trade name: Amberlyst MSPS2-1⋅DRY, manufactured by ORGANO CORPORATION] was added and the mixture was stirred at room temperature for 1 hour. Next, the solution was passed through Celite and a filter with a pore size of 1 μm, and was concentrated under reduced pressure by an evaporator to obtain a target random copolymer (copolymer C). The composition ratio, weight average molecular weight (Mw), and molecular weight distribution (Mw/Mn) of the resulting copolymer C are shown in Table 1.

A 1% by mass aqueous solution of the resulting copolymer C was prepared, and the solubility into water at 25° C. was visually checked. As a result, the aqueous solution was divided into an aqueous phase and a polymer phase, which confirmed insolubilization in water due to introduction of a hydrophobic unit.

### Synthetic Examples 2 to 14

Copolymers A, B, D to N shown in Table 1 were produced by performing synthesis based on Synthetic Example 1 while using EOEOVE or TEGVE as a hydrophilic vinyl ether and using NBVE or tricyclodecanyl vinyl ether (TCDVE) as a hydrophobic vinyl ether and varying the amount of the initiator species and the composition ratio. The resulting copolymers were evaluated for the solubility in water by the same operation as in Synthetic Example 1. The composition ratio, molecular weight (Mw), molecular weight distribution (Mw/Mn), and solubility in water of each copolymer are shown in Table 1.

### Synthetic Examples 15 to 17

Homopolymers O, P, and Q shown in Table 1 were produced by polymerizing each of EOEOVE, NBVE, and TCDVE alone based on Synthetic Example 1. The resulting homopolymers were evaluated for the solubility in water by the same operation as in Synthetic Example 1. The evaluation results of the molecular weight (Mw), molecular weight distribution (Mw/Mn), and solubility in water of each homopolymer are shown in Table 1.

TABLE 1

| | | Hydro- | | | |
|---|---|---|---|---|---|
| Sample | Chemical | philic unit | | Mw/ | Solubility in |
| name | name | (mol %) | Mw | Mn | water (25° C.) |
| A | NBVE-ran- | 10 | 3400 | 1.43 | Insoluble |
| B | EOEOVE | | 4200 | 1.48 | Insoluble |
| C | | | 11500 | 1.12 | Insoluble |
| D | | | 14700 | 1.11 | Insoluble |
| E | | | 24300 | 1.16 | Insoluble |
| F | | 20 | 3600 | 1.20 | Insoluble |
| G | | | 11700 | 1.14 | Insoluble |
| H | | | 14100 | 1.10 | Insoluble |
| I | | | 23300 | 1.08 | Insoluble |
| J | NBVE-ran- | 10 | 9900 | 1.10 | Insoluble |
| | TEGVE | | | | |
| K | NBVE-block- | 10 | 14600 | 1.12 | Insoluble |
| | TEGVE | | | | |
| L | TCDVE-ran- | 30 | 35200 | 1.15 | Insoluble |
| | TEGVE | | | | |
| M | TCDVE-block- | 30 | 44100 | 1.09 | Insoluble |
| | TEGVE | | | | |
| N | TEGVE-block- | 30 | 11700 | 1.14 | Insoluble |
| | NBVE- | | | | |
| | block-TEGVE | | | | |
| O | EOEOVE | 100 | 11600 | 1.21 | Soluble |
| P | NBVE | 0 | 25200 | 1.14 | Insoluble |
| Q | TCDVE | 0 | 29600 | 1.08 | Insoluble |

### (Solubility in Water)

Insoluble: divided into aqueous phase and polymer phase.
Soluble: uniformly dissolved.

### Examples 1 to 14 and Comparative Examples 1 to 3

### Blood Compatibility Test:

In order to test blood compatibility, polyethylene terephthalate (PET) plates (Examples 1 to 14) surfaces of which were coated with the respective copolymers A to N according to the Synthetic Examples, and PET plates (Comparative Examples 1 to 3) surfaces of which were coated with the respective homopolymers O to Q as comparative examples were subjected to a platelet adhesion test.

The surface coating of the PET plates with polymers according to Examples and Comparative Examples was performed by applying a 0.2 wt/vol % toluene solution of each polymer obtained in the Synthetic Examples on a surface of the PET plate and evaporating the solvent for exsiccation.

0.2 mL of a human fresh platelet-rich plasma treated for anti-coagulation with sodium citrate was added dropwise with a pippete to the PET surface coated with each polymer and the PET plate coated with no polymer (blank), and the plates were allowed to stand at 37° C. for 60 minutes. Subsequently, the plates were rinsed with a phosphate buffer solution and were fixed with glutaraldehyde. Then, the sample surfaces were each observed by a scanning electron microscope to count the number of platelets that adhered in an area of 1×104 μm². Although peeling of a coating layer was observed in the case of the polymer O (homopolymer of EOEOVE), the evaluation was continued as it was.

The results of the blood compatibility test are shown in Table 2. It was shown that the PET plate a surface of which was coated with each of the copolymers A to N provides a smaller number of platelet adhesion as compared with Comparative Examples and the blank PET plate.

TABLE 2

| | | | | Platelet |
|---|---|---|---|---|
| | | | Hydro- | adhesion |
| | Sample | Chemical | philic unit | (×10⁵ |
| Example | name | name | (mol %) | cells/cm2) |
| Example 1 | A | NBVE-ran- | 10 | 0.32 |
| Example 2 | B | EOEOVE | | 0.06 |
| Example 3 | C | | | 0.05 |
| Example 4 | D | | | 0.30 |
| Example 5 | E | | | 0.06 |
| Example 6 | F | | 20 | 0.05 |
| Example 7 | G | | | 0.20 |
| Example 8 | H | | | 0.50 |
| Example 9 | I | | | 2.00 |
| Example 10 | J | NBVE-ran- | 10 | 0.20 |
| | | TEGVE | | |
| Example 11 | K | NBVE-block- | 10 | 0.83 |
| | | TEGVE | | |
| Example 12 | L | TCDVE-ran- | 30 | 0.20 |
| | | TEGVE | | |
| Example 13 | M | TCDVE-block- | 30 | 1.03 |
| | | TEGVE | | |
| Example 14 | N | TEGVE-block- | 30 | 0.50 |
| | | NBVE- | | |
| | | block-TEGVE | | |
| Comparative | O | EOEOVE | 100 | 6.70 |
| Example 1 | | | | |
| Comparative | P | NBVE | 0 | 2.80 |
| Example 2 | | | | |
| Comparative | Q | TCDVE | 0 | 6.80 |
| Example 3 | | | | |
| Blank | PET base | — | — | 9.75 |

### Examples 15 to 18

### Cancer Cell Adhesion Test:

PET plates coated with the copolymers A, B, and C were fabricated in the same manner as in Examples 1 to 3 and 1.0 mL of a cancer cell suspension (prepared at 10,000 cells/mL in a medium having serum added at 10%) was added with a pippete to PET surfaces coated with the copolymers and a PET plate coated with no copolymer (blank), and the plates were allowed to stand at 37° C. for 60 minutes. As the cancer cells, a human fibrosarcoma cell line, HT-1080 was used. Subsequently, the plate was rinsed with a physiological buffer saline solution and the number of cells adhered on the sample surface was counted. For facilitating the counting, the cells were fixed with formaldehyde, then the cell nuclei were stained with 4′,6-diamino-2-phenylindole (DAPI), and the number of the cell nuclei was counted using confocal laser scanning microscope (Olympus FV-1000) and the count was taken as the cell number.

The results of the cell adhesion test are shown in Table 3. Although the copolymers A, B, and C contain hydrophobic units at 90% by mole, the cell adhesion numbers thereof were found to be very small.

TABLE 3

| | | | | | HT1080 |
|---|---|---|---|---|---|
| | | Hydro- | | | adhesion |
| Sample | Chemical | philic unit | | Mw/ | (×10⁴ |
| name | name | (mol %) | Mw | Mn | cells/cm2) |
| A | NBVE-ran- | 10 | 3400 | 1.43 | 0.03 |
| B | EOEOVE | | 4200 | 1.48 | 0.19 |
| C | | | 11500 | 1.12 | 0.03 |
| PET base | — | — | — | — | 1.30 |

### INDUSTRIAL APPLICABILITY

As described above, coating films formed of the copolymer of the present invention are excellent in film formation properties and resistance to water dissolution, and can prevent adhesion of platelets, and can prevent production of thrombi caused in turn by the adhesion. Thus, the copolymer of the present invention is useful as a biocompatible material conforming to IS010993.

In addition, an antithrombotic coating film formed by using the antithrombotic coating agent of the present invention containing the copolymer of the present invention has excellent antithrombotic properties, and particularly when the antithrombotic coating film is formed on a medical device that is brought into contact with blood, production of thrombi can be prevented.

Furthermore, a coating film formed of the copolymer of the present invention can prevent adhesion of biocomponents, such as cells, and can be suitably used as a material having anti-fouling properties in various medical devices.

Accordingly, the present invention is highly useful in the medical field and the field of production of medical devices.

1: A copolymer, comprising:
a repeating unit (A) represented by formula (1): 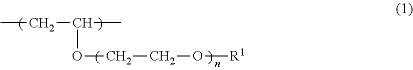
wherein R¹ represents a methyl group or an ethyl group, and
n represents an integer of 2 to 10, and
a repeating unit (B) represented by formula (2): 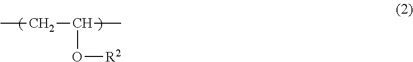
wherein R² represents an aliphatic hydrocarbon group.

2: The copolymer according to claim 1, wherein the composition ratio (molar ratio) of the repeating unit (A) to the repeating unit (B) is 90/10 to 1/99.

3: The copolymer according to claim 1, wherein R² is a linear or branched alkyl group or alkenyl group having 2 to 10 carbon atoms or a monocyclic or polycyclic alkyl group or alkenyl group having 5 to 15 carbon atoms.

4: The copolymer according to claim 1, wherein R² is a linear or branched alkyl group or alkenyl group having 2 to 10 carbon atoms.

5: A biocompatible material, comprising:
the copolymer according to claim 1.

6: An antithrombotic coating agent, comprising:
the copolymer according to claim 1 and
an organic solvent.

7: The antithrombotic coating agent according to claim 6, wherein the organic solvent is at least one selected from the group consisting of an aromatic hydrocarbon, an aliphatic hydrocarbon, a halogenated hydrocarbon, an ether, an alcohol, a ketone, and an ester.

8: The antithrombotic coating agent according to claim 6, wherein the copolymer is present from 0.05 to 10 parts by mass and
the organic solvent is present from 99.95 to 90 parts by mass.

9: An antithrombotic coating film, comprising:
the copolymer according to claim 1.

10: A medical device provided with an antithrombotic coating film formed of the copolymer according to claim 1.

11: The medical device according to claim 10, wherein a part or the whole of a section to be in contact with blood is coated with the antithrombotic coating film.

12: The medical device according to claim 10,
wherein the medical device is an intracorporeal implant type artificial organum or therapeutic implement, an extracorporeal circulation type artificial organ, a catheter, a blood vessel prosthesis, a vascular bypass tube, a prosthetic valve, a blood filter, a plasmapheresis device, a blood transfusion apparatus, or an extracorporeal circuit for blood.
